(19) United States
(12) Patent Application Publication
(10) Pub. No.: US 2022/0288045 A1
(43) Pub. Date: Sep. 15, 2022

(54) Stable Pharmaceutical Composition Comprising Esomeprazole And Sodium Bicarbonate

(71) Applicant: Chong Kun Dang Pharmaceutical Corp., Seodaemun-gu, Seoul (KR)

(72) Inventors: Jong Seo Choi, Yongin-si Gyeonggi-do (KR); Min Soo Kim, Yongin-si Gyeonggi-do (KR); Shin Jung Park, Yongin-si, Gyeonggi-do (KR); Jong Lae Lim, Yongin-si Gyeonggi-do (KR)

(73) Assignee: Chong Kun Dang Pharmaceutical Corp., Seodaemun-gu, Seoul (KR)

(21) Appl. No.: 17/630,261

(22) PCT Filed: Jul. 14, 2020
(86) PCT No.: PCT/KR2020/009250

§ 371 Date: Jan. 26, 2022

(30) Foreign Application Priority Data
Jul. 26, 2019 (KR) 10-2019-0091266

(51) Int. Cl. A61K 31/4439; A61K 9/20; A61K 33/00

(52) U.S. Cl. CPC A61K 31/4439; A61K 9/2081; A61K 33/00

(57) ABSTRACT

The present invention relates to a stable pharmaceutical composition comprising an omeprazole, an enantiomer thereof or a pharmaceutically acceptable salt thereof, and sodium bicarbonate. Specifically, the present invention relates to a pharmaceutical composition having improved stability, which includes a low content of sodium bicarbonate and has excellent dissolution rate and bioavailability and a reduced side effect due to a high content of sodium bicarbonate.

### TECHNICAL FIELD

The present invention relates to a stable pharmaceutical composition comprising an omeprazole, an enantiomer thereof or a pharmaceutically acceptable salt thereof, and sodium bicarbonate. Specifically, the present invention relates to a pharmaceutical composition having improved stability, which includes a low content of sodium bicarbonate and has excellent dissolution rate and bioavailability and a reduced side effect due to a high content of sodium bicarbonate.

### BACKGROUND ART

The chemical name for omeprazole is 5-methoxy-24(4-methoxy-3,5-dimethyl-2-pyridinyl)methyllsulfinyl-1H-benzimidazole. Omeprazole exists as two isomers, that are, R-isomer and S-isomer. It is known that S-isomer is superior to R-isomer in terms of therapeutic effect and side effect. The S-isomer is (S)-5-methoxy-24(4-methoxy-3,5-dimethyl-2-pyridinyl)-methyllsulfinyl-1H-benzimidazole, and is generally called esomeprazole. Esomeprazole is a representative proton pump inhibitor(PPI) used in the treatment of dyspepsia, peptic ulcer disease, gastroesophageal reflux disease, and Zollinger-Ellison syndrome), etc.

It is well known in the art that omeprazole, particularly esomeprazole, is susceptible to decomposition or transformation in acidic and neutral media, and more specifically, the decomposition half-life of esomeprazole is known to be less than 10 minutes in aqueous solutions with 3 or less of a pH value. Therefore, the decomposition of esomeprazole is promoted by acidic compounds and is also affected by moisture, heat, organic solvents and light.

Therefore, there has been a lot of demand for a stable esomeprazole formulation, and in order to solve the stability problem, Korean Patent No. 384960 discloses a method of preparing a pellet containing esomeprazole magnesium salt, performing enteric coating it, and then adding an excipient to formulate a tablet. The formulation prepared by the above method is currently on the market under the trade name of Nexium.

However, because enteric-coated tablets such as Nexium are designed not to be immediately absorbed in the stomach but to be dissolved and absorbed in the intestine, they are not suitable for the treatment of diseases requiring immediate therapeutic effect after administration, such as gastric acid-related diseases.

Korean Patent No. 1104349 discloses enteric-coated tablets and capsules improved in term of the problems of stability and physical properties of omeprazole by preparing a solid dispersion formulation using magnesium oxide and povidone.

Korean Patent Publication No. 10-1996-0003605 discloses a method for preparing a solid dispersion formulation by using omeprazole as an active ingredient and adding beta-cyclodextrin and sodium hydroxide as stabilizing ingredients. However, the invention described in the above patent has a problem of using sodium hydroxide, which is harmful to the human body. Since the process of preparing a solid dispersion involves the process of dissolving the active ingredient omeprazole in a solvent, a special stabilizer such as sodium hydroxide is required to stabilize omeprazole during this process.

In order to solve these problems, Korean Patent No. 679767 discloses the process using of a buffering agent such as sodium bicarbonate to omeprazole.

However, when a large amount of sodium bicarbonate is used, there are the disadvantages of reducing the efficacy of omeprazole and causing side effects. Specifically, the administration of a large amount of sodium bicarbonate may aggravate pain in critical patients due to gastric distension, and absorption of sodium bicarbonate may cause belching, which causes upward movement of gastric acid, there is a possibility of exacerbating gastroesophageal reflux disease. In addition, since patients with symptoms such as high blood pressure or heart failure should suppress the intake of sodium that can cause hypertension symptoms, it is not suitable to administer a large amount of sodium bicarbonate to patients with these symptoms. In addition, administration of a large amount of sodium bicarbonate to patients with various complications is at risk of inducing metabolic alkalosis. In addition, since buffers that change gastric and urinary pH may affect to drug absorption, distribution, and metabolic processes, various cautions are required when using a large amount of sodium bicarbonate with omeprazole.

### PRIOR ART LITERATURE

### Patent Literature

(Prior art 1) Korean Patent No. 384960

(Prior art 2) Korean Patent No. 1104349

(Prior art 3) Korean Patent Publication No. 10-1996-0003605

(Prior art 4) Korean Patent No. 679767

### DETAILED DISCLOSURE

### Technical Problem

The present inventors developed a formulation containing sodium bicarbonate for stabilization of omeprazole, which is unstable at low pH. In order to solve the problem caused by that a large amount of sodium bicarbonate was used to increase the pH value in the stomach, the present inventors have develop the pharmaceutical composition with excellent dissolution rate and bioavailability with using a low content of sodium bicarbonate to complete the present invention.

### Technical Solution

The present invention relates to a pharmaceutical composition with improved stability, comprising an omeprazole, an enantiomer thereof or a pharmaceutically acceptable salt thereof, and sodium bicarbonate.

The enantiomer of omeprazole may be S-isomer or R-isomer, but esomeprazole, which is S-isomer, is preferable.

The “pharmaceutically acceptable salt” of the present invention may be a metal salt such as sodium, potassium, calcium, magnesium, zinc, lithium, etc., or an ammonium salt, etc., but is not limited thereto. Among these, a magnesium salt is preferable.

The omeprazole, the enantiomer thereof, or the pharmaceutically acceptable salt thereof may be a solvate thereof, and the solvate may include hydrates such as monohydrate, dihydrate, trihydrate, etc., and may be in an amorphous or crystalline form.

The pharmaceutical composition of the present invention may contain 15 to 50 weights, preferably 20 to 40 weights, of sodium bicarbonate, based on 1 weight of omeprazole of the omeprazole, the enantiomer thereof or the pharmaceutically acceptable salt thereof.

The present invention relates to a pharmaceutical composition wherein the pharmaceutical composition comprises the omeprazole, the enantiomer thereof or the pharmaceutically acceptable salt thereof, and sodium bicarbonate, and contains 20 mg or 40 mg of the omeprazole, the enantiomer thereof or the pharmaceutically acceptable salt thereof and 600 to 1000 mg of sodium bicarbonate, based on weight of omeprazole.

When the sodium bicarbonate content is 600 mg or more, the pH of gastric juice can be made into a neutral environment to inhibit the decomposition of omeprazole, and when 1,000mg or more, there is little change in the pH of gastric juice.

Preferably, the sodium bicarbonate may be 700 to 900 mg, more preferably 800 mg.

The present invention relates to a pharmaceutical composition characterized in that the pharmaceutical composition comprises an esomeprazole or a pharmaceutically acceptable salt thereof, and sodium bicarbonate, and contains 40 mg of esomeprazole based on the weight of esomeprazole and 800 mg of sodium bicarbonate, characterized in that when the pharmaceutical composition is administered, the time to reach the maximum blood concentration of the esomeprazole or the pharmaceutically acceptable salt thereof is within 1 hour. The esomeprazole or the pharmaceutically acceptable salt thereof may be in the form of pellets or granules. The pellets or granules may be coated with a coating agent.

In case of single administration of the composition of the present invention, the time to reach the maximum blood concentration of the esomeprazole or the pharmaceutically acceptable salt thereof may be within 1.5 hours. Preferably, it may be within 1 hour.

In case of repeated administration of the composition of the present invention, the time to reach the maximum blood concentration of the esomeprazole or the pharmaceutically acceptable salt thereof may be within 1.25 hours. Preferably, it may be within 1 hour.

In the present invention, “pellet” can be prepared by spraying a coating solution containing an active ingredient or excipient on spherical sucrose.

In the present invention, “granule” may be prepared by using a wet granulation method with a binder solution or a dry granulation method without a binder solution.

Preferably, the esomeprazole or the pharmaceutically acceptable salt thereof is esomeprazole magnesium salt, and more preferably, esomeprazole magnesium salt trihydrate.

Sodium bicarbonate contained in the composition of the present invention may be in the form of wet granules.

When administering the composition of the present invention, the time for maintaining 4 or less of the intragastric pH for 24 hours after administration compared to the time for maintaining 4 or less of the intragastric pH for 24 hours before administration can be reduced by 50% or more.

The composition of the present invention can increase intragastric pH within 50 minutes after a single administration. In addition, the composition of the present invention may increase the intragastric pH within 30 minutes after repeated administration.

The composition of the present invention may have 80% or more of the 24-hour integrated gastric acidity reduction rate (%) after oral administration.

The composition of the present invention may be formulated as pellets, capsules, tablets (including single-layered tablets, double-layered tablets, inner core tablets, etc.), granules, etc., but is not limited thereto. Preferably, the formulation of the present invention is a tablet.

The formulation according to the present invention may be prepared according to a method for preparing any oral solid formulation known in the art, specifically granules, pellets, capsules, or tablets.

Specifically, the present invention relates to the method for preparing the pharmaceutical formulation comprising:

(a) coating a core with a primary coating solution containing an esomeprazole or a pharmaceutically acceptable salt thereof to prepare the primary coating product;

(b) coating the primary coating product with a secondary coating solution containing a coating agent to prepare the secondary coating product;

(c) mixing the secondary coating product with sodium bicarbonate to obtain a mixture;

(d) tabletting the mixture to obtain a bare tablet; and

(e) coating the bare tablet with a tertiary coating solution and then drying to obtain a coated tablet.

In one embodiment, the sodium bicarbonate of step (c) is mixed with the coating product after wet granulation. In another embodiment, the core may be spherical sucrose.

The coating agent may be one or more selected from the group consisting of polyvinyl alcohol, povidone, hydroxypropyl cellulose, hydroxypropyl methyl cellulose, polyvinyl acetate, ethyl cellulose, and dimethylaminoethyl methacrylate/methyl methacrylate copolymer, but is not limited thereto.

The present invention also provides a method for preparing the pharmaceutical formulation comprising:

(a) wet-granulating or dry-granulating of an esomeprazole or a pharmaceutically acceptable salt thereof to obtain granules;

(b) mixing the granules with sodium bicarbonate to obtain a mixture;

(c) tabletting the mixture to obtain a bare tablet; and

(d) coating the bare tablet with a coating solution and then drying to obtain a coated tablet.

In one embodiment, the granules of step (a) may contain sodium bicarbonate, wherein the sodium bicarbonate may be contained 0 to 75% by weight, preferably 50% by weight or less, more preferably 30% by weight or less, on the total weight of sodium bicarbonate contained in the formulation.

In one embodiment, the sodium bicarbonate of step (b) is mixed with the granules after wet granulation.

### ADVANTAGEOUS EFFECTS

The present invention relates to a pharmaceutical composition with improved stability, comprising an omeprazole, an enantiomer thereof or a pharmaceutically acceptable salt thereof, and sodium bicarbonate. The pharmaceutical composition of the present invention has improved stability, has excellent dissolution rate and bioavailability by containing a small amount of sodium bicarbonate, and has the reducing effect of side effects. In addition, in a pharmaceutical composition prepared by wet-granulating of sodium bicarbonate and then mixing with esomeprazole pellets or granules, it shows effects of increasing the dissolution rate of esomeprazole.

### BRIEF DESCRIPTION OF DRAWINGS

FIGS. 1 and 2 show the concentration of esomeprazole at the single administration and the repeated administration of the test drug (40/800 mg and 40/900 mg tablets) according to Example 3 and the reference drug (Nexium tablet).

FIGS. 3 and 4 are graphs showing the esomeprazole dissolution rate of tablets prepared by wet granulating of sodium bicarbonate and then mixing with esomeprazole pellets and tabletting, and tablets prepared by simple mixing of sodium bicarbonate with esomeprazole pellets.

FIGS. 5 and 6 show the results of 24-hour pH monitoring at the single administration and the repeated administration of the test drug (40/800 mg tablet) according to Example 6 and the reference drug (Nexium tablet (D026 40 mg)).

### MODE FOR THE INVENTION

Hereinafter, the present invention will be more specifically described through working examples. However, these examples are provided only for the purpose of illustration to help the understanding of the present invention, but the scope of the present invention is not limited by the following working examples.

### [Example 1] Preparation of Esomeprazole Tablets Containing 800mg Sodium Bicarbonate

A tablet containing esomeprazole and sodium bicarbonate was prepared by the following method.

1. Primary Pellet Coating

After adding and dissolving in purified water and hydroxypropyl cellulose, arginine, simethicone, esomeprazole magnesium trihydrate (40 mg as esomeprazole), magnesium oxide and talc were added and dispersed to prepare a primary coating solution. Spherical sucrose was put in a fluidized bed coater and the primary coating solution was sprayed to perform the primary pellet coating process (the primary coating product).

2. Secondary Pellet Coating

Purified water, polyvinyl alcohol, talc, titanium oxide, glycerol monocaprylocaprate and sodium lauryl sulfate were put in a preparation tank and dispersed to prepare a secondary coating solution. The primary coating product was put in the fluidized bed coater, and the secondary coating solution was sprayed to perform the secondary pellet coating process (the secondary coating product).

3. Post-Mixing (Simple Mixing Method)

The secondary coating product was put in a mixer and sodium bicarbonate (800 mg) was added. In this case, purified water may be included depending on the moisture content. In addition, copovidone, crospovidone and sodium stearyl fumarate were added and mixed (the final mixture).

4. Tableting

The final mixture was compressed with a tablet press (a bare tablet).

5. Tertiary coating

Polyvinyl alcohol, talc, titanium oxide, glycerol monocaprylocaprate, sodium lauryl sulfate, red iron oxide, black iron oxide, yellow iron oxide and purified water were added to the preparation tank and dissolved to prepare a tertiary coating solution. The bare tablet was put in a coater, coated with the tertiary coating solution, and then dried to obtain a final coated tablet.

### [Example 2] Preparation of Esomeprazole Tablet Containing 900 mg Sodium Bicarbonate

An esomeprazole tablet was prepared in the same manner as in Example 1, except that 900 mg of sodium bicarbonate in step 3 of Example 1 was used.

### [Example] 3 Preparation of Esomeprazole Tablet Containing 800 mg of Wet-Granulated Sodium Bicarbonate

A tablet containing esomeprazole and sodium bicarbonate was prepared by the following method.

1. Primary Pellet Coating

After adding and dissolving in purified water and hydroxypropyl cellulose, arginine, simethicone, esomeprazole magnesium trihydrate (40 mg as esomeprazole), magnesium oxide and talc were added and dispersed to prepare a primary coating solution. Spherical sucrose was put in a fluidized bed coater and the primary coating solution was sprayed to perform the primary pellet coating process (the primary coating product).

2. Secondary Pellet Coating

Purified water, polyvinyl alcohol, talc, titanium oxide, glycerol monocaprylocaprate and sodium lauryl sulfate were put in a preparation tank and dispersed to prepare a secondary coating solution. The primary coating product was put in the fluidized bed coater, and the secondary coating solution was sprayed to perform secondary pellet coating process (the secondary coating product).

3. Post-Mixing (Wet Granulation Method)

After preparing a binder solution with copovidone and water in a separate container, it was combined with sodium bicarbonate (800 mg) and dried to prepare a combined product. Then, the combined product and the secondary coating product were put in a mixer, and copovidone, crospovidone and sodium stearyl fumarate were added and mixed (the final mixture).

4. Tableting

The final mixture was compressed with a tablet press. (a bare tablet)

5. Tertiary Coating

Polyvinyl alcohol, talc, titanium oxide, glycerol monocaprylocaprate, sodium lauryl sulfate, red iron oxide, black iron oxide, yellow iron oxide and purified water were added to the preparation tank and dissolved to prepare a tertiary coating solution. The bare tablet was put in a coater, coated with the tertiary coating solution, and then dried to obtain a final coated tablet.

### [Example 4] Preparation of Esomeprazole Tablets Containing 900 mg Wet-Granulated Sodium Bicarbonate

An esomeprazole tablet was prepared in the same manner as in Example 3, except that 900 mg of sodium bicarbonate in step 3 of Example 3 was used.

### Example 5

The formulation of Example 5 (40 mg of esomeprazole, 800 mg of sodium bicarbonate) was prepared according to the following preparation method.

1. Mixing

Esomeprazole magnesium trihydrate and microcrystalline cellulose were added and mixed with a High Speed Mixer.

2. Preparation of the First Mixing Part (Wet Granules)

Hydroxypropyl cellulose was added and dissolved in purified water to prepare a binding solution. The binding solution was added to the mixture, and the mixture was combined and dried to prepare wet granules in the first mixing part.

3. Preparation, Mixing and Lubrication of the Second Mixing Part The wet granules of the first mixing part, sodium bicarbonate, copovidone and croscarmellose sodium were put in a mixer and mixed, and then sodium stearyl fumarate was added and lubricated to prepare a final mixture. At this time, the part excluding the first mixing part forms the second mixing part.

4. Tableting and Coating The final mixture was tableted with a tablet press (a bare tablet). Polyvinyl alcohol, titanium oxide, polyethylene glycol, talc and purified water were put into the preparation tank and dissolved. The bare tablet was put into a coater, coated, and then dried to obtain a coated tablet.

### Example 6

The formulation of Example 6 (40 mg of esomeprazole, 800 mg of sodium bicarbonate) was prepared according to the following preparation method.

1. Mixing and Lubrication

Esomeprazole magnesium trihydrate, sodium bicarbonate, magnesium oxide and crospovidone were added and mixed, and then sodium stearyl fumarate was added to obtain a lubricated mixture.

2. Preparation of the First Mixing Part (Dry Granules)

The mixture was banged with a sledgehammer to prepare the first mixing part.

3. Preparation, Mixing, and Lubrication of the Second Mixing Part

The first mixing part, sodium bicarbonate, copovidone and crospovidone were added and mixed, and then sodium stearyl fumarate waw added and lubricated to prepare a final mixture. In this case, the part excluding the first mixing part forms the second mixing part.

4. Tableting and Coating

The final mixture was tableted with a tablet press (a bare tablet). Hydroxypropylmethylcellulose, titanium oxide, polyethylene glycol and purified water were put into the preparation tank and dissolved. The bare tablet was put into a coater, coated, and then dried to obtain a coated tablet.

### Example 7

1. Preparation of the First Mixing Part

Esomeprazole magnesium trihydrate, mannitol, copovidone, crospovidone and sodium stearyl fumarate were uniformly mixed to prepare the first mixing part.

2. Preparation of the Second Mixing Part

Sodium bicarbonate, copovidone, croscarmellose sodium, magnesium stearate and sodium stearyl fumarate were uniformly mixed to prepare the second mixing part.

3. Tableting

The first mixing part and the second mixing part were tableted with a tablet press (bare tablet)

4. Coating

Polyvinyl alcohol, titanium oxide, polyethylene glycol, talc and purified water were put into the preparation tank and dissolved. The bare tablet was put into a coater, coated, and then dried to obtain a coated tablet.

### Example 8 and 9

According to the preparation method of Example 7 above, the formulations of Examples 8 and 9 were prepared, but sodium bicarbonate was additionally mixed in the first mixing part preparation process of the first step of the preparation method to be included sodium bicarbonate in the first mixing part and the second mixing part.

The first mixing parts of Examples 8 and 9 contained 5 and 10% by weight of sodium bicarbonate based on the weight of sodium bicarbonate (800 mg) of the entire formulation, respectively.

### Examples 10 to 14

According to the preparation method of Example 5 above, the formulations of Examples 10 to 14 were prepared, but sodium bicarbonate was additionally mixed in the mixing process of the first step of the preparation method to be included sodium bicarbonate to the first mixing part and the second mixing part. The first mixing parts of the formulations of Examples 8 to 12 contained 10, 30, 40, 50 and 75% by weight of sodium bicarbonate based on the weight of sodium bicarbonate (800 mg) of the entire formulation, respectively.

### [Experimental Example 1] Stability Test of Esomeprazole and Omeprazole According to pH

After adding each 2 mL of 20 mg/mL strength of esomeprazole and omeprazole solutions to 100 mL of buffer solution, the content according to pH was analyzed, and the analysis method is as follows.

<Analysis Method>

A) Detector: Ultraviolet visible light absorption spectrometer (measurement wavelength: 280 nm)

B) Column: Inertsil C8-3 (4.6×150 mm, 5 μm) or equivalent column

C) Injection amount: 20 μl

D) Flow rate: 1.5 mL/min

E) Column temperature: constant temperature around 40° C.

F) Sample temperature: constant temperature around 10° C.

G) Analysis time: 6 minutes

H) Mobile phase: pH 7.6 buffer and acetonitrile mixture (65:35) pH 7.6 buffer above is the solution prepared by weighing 0.725 g of sodium hydrogen phosphate monohydrate (NaH₂PO₄.H₂O) and 4.472 g of disodium hydrogen phosphate anhydrous (Na₂HPO₄) and putting in a 1 L volumetric flask, dissolving in purified water, and then taking 250 mL of the marked solution to place to a 1 L volumetric flask, and marking with purified water, and adjusting to pH 7.6 with phosphoric acid.

The analysis results are shown in Table 1 below.

TABLE 1

| | | 5 | 10 | 15 | 30 | 45 | 60 | 120 |
|---|---|---|---|---|---|---|---|---|
| | | min | min | min | min | min | min | min |
| pH4.0 | S-omeprazole | 64.1 | 31.0 | 15.9 | 6.9 | 1.7 | 0.9 | 0.1 |
| | Omeprazole | 57.2 | 29.4 | 15.3 | 6.7 | 1.6 | 0.8 | 0.1 |
| pH6.0 | S-omeprazole | 74.4 | 72.8 | 71.0 | 68.9 | 66.3 | 63.6 | 54.9 |
| | Omeprazole | 74.9 | 74.2 | 71.7 | 69.6 | 65.4 | 64.1 | 55.1 |
| pH6.8 | S-omeprazole | 95.6 | 90.8 | 90.5 | 89.7 | 88.9 | 88.3 | 86.2 |
| | Omeprazole | 91.5 | 90.5 | 89.9 | 89.8 | 88.8 | 88.3 | 85.5 |
| pH7.0 | S-omeprazole | 99.6 | 99.0 | 99.3 | 98.9 | 98.7 | 98.1 | 96.8 |
| | Omeprazole | 100.5 | 99.0 | 98.9 | 98.2 | 97.9 | 97.5 | 95.8 |
| pH7.3 | S-omeprazole | 100.1 | 100.1 | 100.0 | 100.0 | 100.0 | 100.0 | 99.90 |
| | Omeprazole | 99.8 | 100.0 | 99.9 | 99.8 | 99.8 | 99.8 | 99.86 |
| pH7.5 | S-omeprazole | 99.4 | 99.3 | 99.2 | 99.1 | 99.3 | 99.1 | 98.2 |
| | Omeprazole | 100.5 | 99.7 | 99.7 | 99.4 | 99.2 | 98.9 | 98.2 |
| pH8.0 | S-omeprazole | — | — | 101.1 | 100.9 | 100.7 | 100.7 | 100.0 |
| | Omeprazole | — | — | 99.7 | 99.6 | 99.5 | 99.5 | 98.6 |

As shown in Table 1, it was confirmed that esomeprazole and omeprazole exhibited stability for at least 2 hours when the pH was 7.0 or higher.

### [Experimental Example 2] pH Confirmation Test of Artificial Gastric Juice According to Sodium Bicarbonate Dosage

To set the content of sodium bicarbonate, the drug release conditions and gastric juice conditions were set as follows. Specifically, 1) the amount of gastric juice on fasting status is generally 20 to 50 mL, 2) the gastric juice secretion is about 2 L/day (about 83 mL/hr), and 3) the total amount of gastric juice reacting with the drug (agent) is about assumed 200 mL, 4) when taking the drug, take it with water, and the amount of water at this time was 200 mL.

Therefore, pH was measured while changing the sodium bicarbonate capacity in a solution (37° C.) in which 200 mL of purified water was added to 200 mL of artificial gastric juice, and the measurement results are shown in Table 2 below.

TABLE 2

| | Sodium bicarbonate (mg) | | | | | | | | |
|---|---|---|---|---|---|---|---|---|---|
| | 500 | 600 | 700 | 800 | 900 | 1,000 | 1,100 | 1,200 | 1,300 |
| pH | 5.77 | 6.51 | 7.30 | 7.30 | 7.31 | 7.37 | 7.38 | 7.38 | 7.40 |

As shown in Table 2 above, as the dosage of sodium bicarbonate increased, the pH value increased, and it was confirmed that the pH had little change at 1,000 mg or more of sodium bicarbonate. In addition, it was confirmed that the amount of sodium bicarbonate that can represent neutral pH by neutralizing 200 mL of artificial gastric juice is at least 600 mg or more.

### [Experimental Example 3] Evaluation of Pharmacokinetic Properties (PK)

A tablet containing 40/800 mg and a tablet containing 40/900 mg of esomeprazole/sodium bicarbonate prepared in Examples 3 and 4, respectively, were used as test drugs (T), and commercially available Nexium Tablet® 40 mg was used as a reference drug (R), and after oral administration of these to the subject, the blood concentration of esomeprazole was measured.

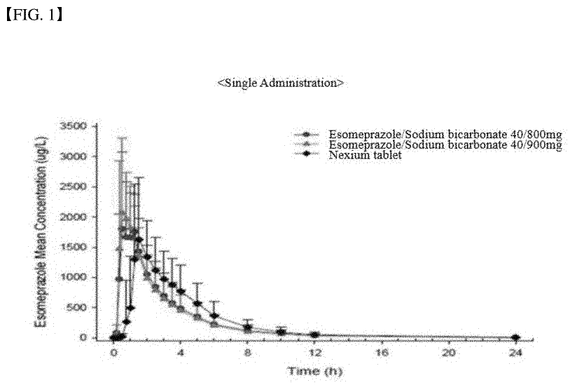

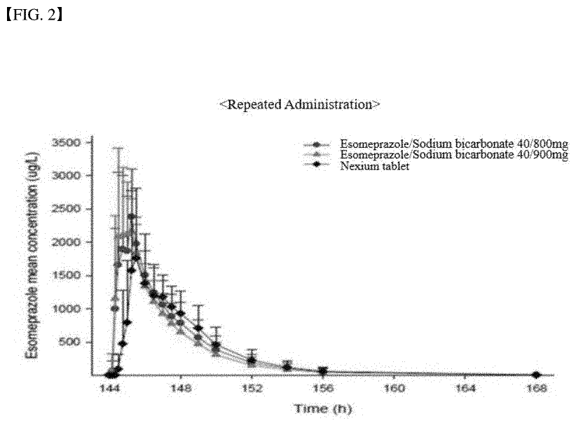

A randomized, washout 7 days or longer, 3×3 crossover clinical trial was performed, subjects per each drug group were 6, and the drug was administered singly/repeatedly. The blood concentration-time curves of esomeprazole obtained for each drug are shown in FIGS. 1 and 2. AUC values were measured from the graphs of FIGS. 1 and 2, and the ratio of each test drug (T) to the reference drug (R) (T/R ratio) and its 90% confidence interval are shown in Table 3.

TABLE 3

| | | T/R | 90% Confidence |
|---|---|---|---|
| Classification | Test Drug | Ratio | Interval |
| Single | 40/800 mg | 0.96 | 0.89-1.02 |
| Administration | 40/900 mg | 0.97 | 0.89-1.06 |
| Repeated | 40/800 mg | 1.13 | 1.07-1.12 |
| Administration | 40/900 mg | 1.01 | 0.96-1.07 |

From the results of Table 3 and FIGS. 1 and 2, it was confirmed that 40/800 mg and 40/900 mg tablets, the test drugs according to the present invention were in almost the same range as AUC of Nexium tablet (that is, the T/R ratio was 0.96 to 1.13). From this, it was confirmed that the test drugs were biologically equivalent to the AUC value of Nexium Tablet, the reference drug.

In particular, considering that the content of commercially available tablets containing esomeprazole/sodium bicarbonate (Trade name: Esoduo®) is 20/800 mg, when esomeprazole is increased to 40 mg, sodium bicarbonate generally is increased as well. However, surprisingly, from the above results, it was confirmed that the 40/800 mg tablet of the present invention exhibited almost the same AUC value as the 40/900 mg tablet, and had bioequivalence with the reference drug.

From the above results, the tablet of the present invention can use at low content without increasing the amount of sodium bicarbonate even when esomeprazole is increased (for example, 2 times), so that it was confirmed that excellent dissolution rate and bioavailability were exhibited without side effects due to use of the large amount of sodium bicarbonate.

### [Experimental Example 4] Evaluation of Pharmacodynamic Properties (PD)

In a clinical trial involving 37 subjects, the tablet of Example 3 containing 40/800 mg of esomeprazole/sodium bicarbonate was used as a test drug (T), and after repeated or single oral administration once a day for 7 days, 24-hour Integrated Gastric Acidity was evaluated, and the results are shown in Table 4 below.

TABLE 4
<Integrated Gastric Acidity Reduction Rate (%) after
Single/Repeated Administration>

| | Number of | Integrated Gastric Acidity |
|---|---|---|
| | patients | Reduction Rate (%) |
| Repeated | 37 | 90.01 |
| Administration | | |
| Single | 36 | 87.15 |
| Administration | | |

As shown in Table 4, it was confirmed that the integrated gastric acidity reduction rate (%) from baseline for 24 hours after repeated oral administration of the test drug for 7 days is about 90%, and the integrated gastric acidity reduction rate after single oral administration is about 87%.

### [Experimental Example 5] Evaluation of Dissolution Rate of Esomeprazole According to the Preparation Method of Sodium Bicarbonate

The content of sodium bicarbonate is 800 mg, which occupies the largest proportion of the tablet weight, so the physical properties of sodium bicarbonate affect the release rate of esomeprazole. In this Experimental example, the esomeprazole dissolution rates of a tablet prepared by simply mixing sodium bicarbonate with esomeprazole pellets (Example 1) and a tablet prepared by wet granulating of sodium bicarbonate (Example 3) were compared.

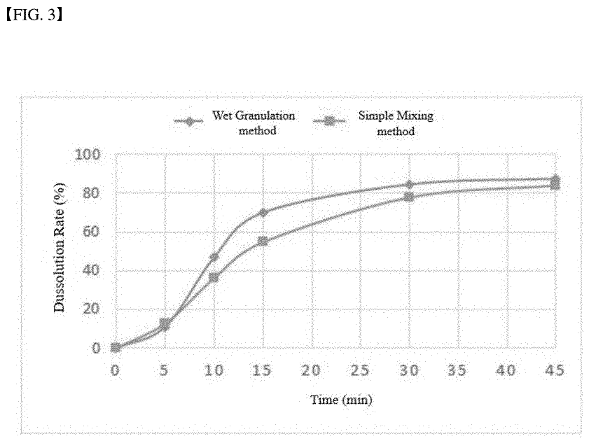

The dissolution rates were compared with the following paddle method conditions, and the results are shown in FIG. 3.

<Dissolution test conditions>

1) Dissolution method: Korean Pharmacopoeia 2nd method (paddle method)

2) Eluate: pH 7.4 solution (a solution dissolved 1.56 g of sodium hydroxide and 6.8 g of potassium dihydrogen phosphate in 1 L purified water), 900 mL

3) Elution temperature: 37 ±0.5° C.

4) Rotation speed: 75 rpm

5) Test time: 45 minutes

<Analysis conditions>

1) Detector: UV absorbance spectrometer (measurement wavelength: 302 nm)

2) Column: Capcell Pak C18 (4.6×150 mm, 5 μm) or equivalent column

3) Injection volume: 20 μl

4) Flow rate: 1.0 mL/min

5) Column temperature: constant temperature around 30° C.

6) Sample temperature: constant temperature around 10° C.

7) Mobile phase: mixture of acetonitrile, pH 7.3 buffer and water (350 : 500: 150)

* pH 7.3 Buffer: A solution in which 0.5 mL of a 1 mol/L sodium dihydrogen phosphate solution and 60 mL of a 0.5 mol/L disodium hydrogen phosphate solution were taken, put in a 1 L volumetric flask, and marked with purified water.
4 method dissolution rate (ng/mL)

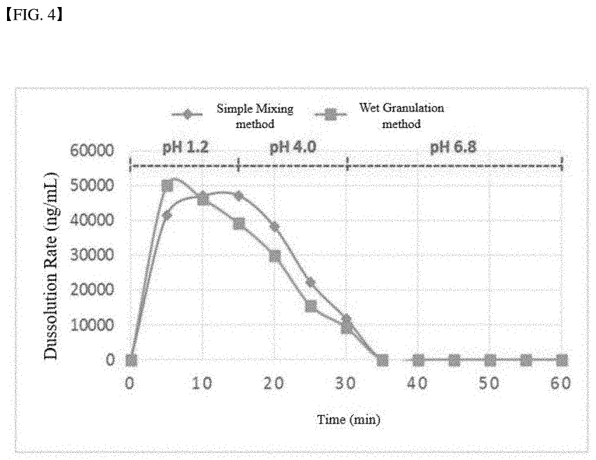

The dissolution rate was compared under the following Flow Through Cell method conditions, and the results are shown in FIG. 4.

<Dissolution test conditions>

1) Dissolution method: Korean Pharmacopoeia 3rd method (Flow Through Cell method)

2) Eluate: pH 1.2→pH 4.0

3) Elution temperature: 37±0.5° C.

4) Flow rate: 2 mL/min

5) Test time: pH 1.2 (15 minutes)→pH 4.0 (15 minutes)

5) Cell size: 22.4 mm

<Analysis conditions>

1) Detector: UV absorbance spectrometer (measurement wavelength: 302 nm)

2) Column: Capcell Pak C18 (4.6×150 mm, 5 μm) or equivalent column

3) Injection volume: 20 μl

4) Flow rate: 1.0 mL/min

5) Column temperature: constant temperature around 30° C.

6) Sample temperature: constant temperature around 10° C.

7) Mobile phase: mixture of acetonitrile, pH 7.3 buffer and water (350:500:150)

* pH 7.3 Buffer: A solution in which 10.5 mL of a 1 mol/L sodium dihydrogen phosphate solution and 60 mL of a 0.5 mol/L disodium hydrogen phosphate solution were taken, put in a 1 L volumetric flask, and marked with purified water.

As shown in FIGS. 3 and 4, in the case of a tablet in which sodium bicarbonate was wet-granulated, it was confirmed that the esomeprazole dissolution rate was higher than that of a tablet in which sodium bicarbonate was simply mixed. This result is because, on simply mixing sodium bicarbonate with esomeprazole pellets, when the tablet is compressed to be within the appropriate Friability criterion (within 0.5%), high compression pressure is applied so that resulting in slow release of the drug. On the other hand, when sodium bicarbonate is wet-granulated and then mixed with esomeprazole pellets to tablet, a relatively low compression pressure enables suitable tableting within the appropriate Friability criterion, so that resulting in rapid release of drug.

### [Experimental Example 6] Clinical Trial of the Combination of Esomeprazole and Sodium Bicarbonate—Measurement of Tmax

After single administration and repeated administration of the tablet of Example 1 (esomeprazole 40 mg/sodium bicarbonate 800 mg), and Nexium Tablet (D026 40 mg) as a reference drug to healthy adults, in order to compare and evaluate pharmacokinetics and pharmacodynamic properties and safety, Clinical trials with randomization, disclosure, repeated administration, and 2×2 crossover design were conducted as shown in Table 5 below.

TABLE 5

| | Number of | 1st | 2nd | |
|---|---|---|---|---|
| Group | Subjects | phase | phase | Washout |
| A | 20 | T | R | At least 7 days |
| | | | | or more |
| B | 20 | R | T | At least 7 days |
| | | | | or more |

T: 1 tablet of Example 1, repeated oral administration once a day for 7 days on fasting status
R: 1 tablet of D026, repeated oral administration once a day for 7 days on fasting status

All subjects take the clinical trial drug (R or T) at the same time in the morning, start the prescribed standard meal (700-800 kcal, 5-25% fat content) after about 1 hour, and finish the meal within 20 minutes.

After baseline 24-hour pH monitoring in the first phase was performed on subjects, from the day 1 of the first phase, the clinical trial drug was administered once a day for a total of 7 days according to each assigned group. All subjects were supposed to start the prescribed standard meal after about 1 hour after administration of the clinical trial drug and finish it within 20 minutes.

After the last dose of the first phase and then washout period of 7 days or more, the subjects were hospitalized again and the second phase clinical trial was performed. On the second clinical trial, the same as the first phase, baseline 24-hour pH monitoring was performed, and then from the day 1 of the second phase, the clinical trial drug was administered once a day for a total of 7 days according to the assigned group. However, unlike in the first phase, the subjects in Group A were administrated the reference drug and the subjects in Group

B were administrated the tablet of Example 1 at a constant time, and the prescribed standard meal was started after about 1 hour after dosing and finished within 20 minutes.

In order to compare the pharmacokinetic properties of the tablet of Example 1 and the reference drug, pharmacokinetic blood collection after single administration was performed on the day 1 of each of the first and second phases, and pharmacokinetic blood collection after repeated administration was performed on the day 7 of each of the first and second phases, carried out immediately before administration and 0.17 (=10 min), 0.33 (=20 min), 0.5, 0.75, 1, 1.25, 1.5, 2, 2.5, 3, 3.5, 4, 5, 6, 8, 10, 12 h after administration (18 times in each phases). The concentration of esomeprazole was measured in plasma separated from the blood collected, and the mean value of the time to reach the maximum blood concentration (Tmax) was analyzed and shown in Table 6 below.

TABLE 6

| Single Administration | | Repeated Administration | |
|---|---|---|---|
| Example 1 | D026 | Example 1 | D026 |
| 0.50 | 1.50 | 0.75 | 1.25 |

In both single administration and repeated administration, the time to reach the maximum blood concentration of the tablet of Example 1 was much shorter than that of the reference drug. Thus, it was confirmed that the tablet of Example 1 can exhibit rapid drug effect by rapidly releasing esomeprazole.

### [Experimental Example 7] Clinical Trial Result of the Combination of Esomeprazole and Sodium Bicarbonate—Measurement of Intragastric pH

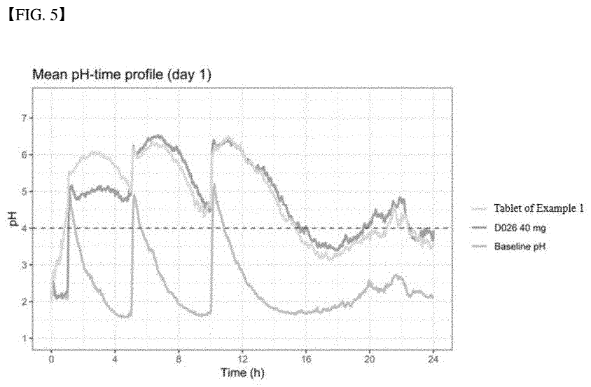

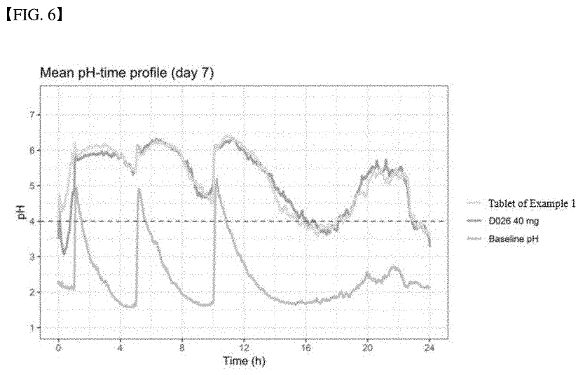

In the clinical trial of Experimental example 6, intragastric pH was measured through 24-hour pH monitoring. 24-hour pH monitoring after single administration was performed on the day 1 of each of the first and second phases, 24-hour pH monitoring after repeated administration was performed on the day 7 of each of the first and second phases, and intragastric pH measurement used MMS Ohmega R pH. The calibratin of pH meter catheter was performed using a standard solution, and only successfully calibrated catheter and pH test device were prepared to use in the 24-hour intragastric pH test. After that, the catheter was sufficiently soaked with lubricating gel or water to reduce the feeling of foreign body, and then inserted into the stomach through the nasal cavity to measure the pH. The results of 24-hour pH monitoring after single administration are shown in FIG. 5, and the results of 24-hour pH monitoring after repeated administration are shown in FIG. 6.

It was confirmed that the pH of the tablet of Example 1 increased from the time point of about 30 minutes onward after single dosing, whereas the pH of the reference drug increased from the time point of 1 hour onward after single dosing.

In addition, it was confirmed that the pH of the tablet of Example 1 increased from the time point of about 20 minutes onward after repeated dosing, whereas the pH of the reference drug gradually increased from the time point of 30 minutes onward after repeated dosing.

As a result, it can be seen that the tablet of Example 1 rapidly raises the intragastric pH as dosing.

### [Experimental Example 8] Clinical Trial Result of the Combination of Esomeprazole and Sodium Bicarbonate—Measurement of Intragastric pH

According to Experimental example 7 above, the time fraction (%) of maintaining pH≤4 in the intragastric pH observed for 24 hours after administration of each clinical trial drug was measured, and the difference by comparing this with the time fraction (%) of maintaining pH≤4 in the intragastric pH observed for 24 hours before drug administration was measured, and as a result, it was confirmed that 54.36% in the case of single administration and 65.81% in the case of repeated administration were decreased.

Therefore, it can be seen that when the tablet of Example 1 is administered, the time for maintaining 4 or less of the intragastric pH is reduced.

1. A pharmaceutical composition
characterized in that the pharmaceutical composition comprises an esomeprazole or a pharmaceutically acceptable salt thereof, and sodium bicarbonate, and contains 40 mg of esomeprazole based on the weight of esomeprazole and 800 mg of sodium bicarbonate,
characterized in that when the pharmaceutical composition is administered, the time to reach the maximum blood concentration of the esomeprazole or the pharmaceutically acceptable salt thereof is within 1 hour.

2. The pharmaceutical composition according to claim 1, characterized in that the esomeprazole or the pharmaceutically acceptable salt thereof is in the form of pellets or granules.

3. The pharmaceutical composition according to claim 2, characterized in that the pellets or granules are coated by coatings.

4. The pharmaceutical composition according to claim 1, characterized in that the esomeprazole or the pharmaceutically acceptable salt thereof is esomeprazole magnesuim.

5. The pharmaceutical composition according to claim 4, characterized in that the esomeprazole magnesium is esomeprazole magnesium trihydrate.

6. The pharmaceutical composition according to claim 1, characterized in that the pharmaceutical composition is in the form of tablet.

7. The pharmaceutical composition according to claim 1, characterized in that the sodium bicarbonate can be present in the form of wet granule.

8. The pharmaceutical composition according to claim 1, characterized in that the time for maintaining 4 or less of the intragastric pH for 24 hours after administration compared to the time for maintaining 4 or less of the intragastric pH for 24 hours before administration of the composition was reduced by 50% or more.

9. The pharmaceutical composition according to claim 1, characterized in that the intragastric pH increases within 50 minutes after single administration of the composition.

10. The pharmaceutical composition according to claim 1, characterized in that the intragastric pH increases within 30 minutes after repeated administration of the composition.

11. The pharmaceutical composition according to claim 1, characterized in that the 24-hour integrated gastric acidity reduction rate (%) after oral administration of the composition is 80% or more.
